(19) United States
(12) Patent Application Publication
(10) Pub. No.: US 2009/0209759 A1
(43) Pub. Date: Aug. 20, 2009

(54) AUTOMATED ITERATIVE DRUG DISCOVERY AND SYNTHESIS

(75) Inventors: Brian Warrington, Hertford (GB); Jeremy Vinter, Hertfordshire (GB); Mark Mackey, Bedfordshire (GB)

Correspondence Address: MARK J. ROSEN P.C., 418 BARCLAY ROAD, BRYN MAWR, PA, 19010, US

(73) Assignee: Cresset Biomolecular Discovery, Ltd., Hertfordshire (GB)

(21) Appl. No.: 12/305,453

(22) PCT Filed: Jun. 18, 2007
(86) PCT No.: PCT/GB2007/050341

§ 371 Date: Dec. 18, 2008

(30) Foreign Application Priority Data
Jun. 19, 2006 (GB) 0612114.9

(51) Int. Cl. C07D 401/06; C07D 401/04; C07D 401/14

(52) U.S. Cl. 544/297; 544/405; 546/274.1

(57) ABSTRACT

The present invention relates to methods and systems for de novo iterative synthesis, an automated iterative drug discovery method and system providing for rapid identification and synthesis of novel compounds.

### FIELD OF THE INVENTION

The present invention relates to methods and systems for automated iterative drug discovery providing for rapid identification of novel compounds which bind to selected targets.

### BACKGROUND OF THE INVENTION

Current methods of drug discovery known in the art suffer from many complications in required materials, speed, cost, difficulty and the like. For example, one such known method relies on a combinatorial chemical library or the members of a directed diversity chemical library.

A combinatorial chemical library is a prechosen plurality of compounds manufactured simultaneously as a mixture. This plurality will have a common structural core and each member will represent a unique configuration of substitution at specific positions on the common core. Most commonly the common core will be attached to a bead structure to facilitate handling. The strategy for preparation is usually one which will lead to a mixture of all possible compound types but any bead will only bear one type of compound. To facilitate identification the compound or the bead or a linker group may contain a coding tag to aid identification. Alternatively, identification may be achieved by mass spectral analysis of the compound after cleavage from the bead. The preparation of a bead-bound (or a solution mixture) combinatorial library is essentially a manual process and for the duration of the library synthesis probably represents the highest level of manual productivity of a chemist. However, library production must be preceded by months of exploratory chemistry to achieve the near perfect yield of each compound required to ensure that unplanned artifacts are not included in the library, then succeeded by a long period of quality assurance to ensure screening results are not misleading. Because of the very limited range of chemistry that can be carried out on solid-supports and the fact that successful library production requires that all members must be synthesizable under the same reaction conditions, it is difficult to use a combinatorial library to create diverse structures. Because of the variable effectiveness of compound cleavage routines, assay concentration is uncertain. In addition, a single concentration does not allow ranking of compound and therefore structure-activity relationships cannot be developed. Essentially, a combinatorial library must be looked on as a slowly produced compound set that gives very limited information on screening. As such, only with great difficulty can it be used in a closed loop manner (i.e. assay results are used to inform the design of a new generation of compounds), and the response cycle time will be impossibly protracted (months to years). The combinatorial library is more a tool for serendipitous discovery of active compounds and even here it represents an unjustifiably dense representation of a minute fraction of chemical space.

A directed diversity library is a prechosen plurality of chemical compounds which are formed by selectively combining a particular set of building blocks in separate reactors. This obviates the need for near perfect yields since individual reaction products can be subjected to individual purification. In addition, quality control and assay issues are eased and the concentration dependence of activity or affinity can be produced to enable the ranking of compound properties. In essence this would be the procedure that a chemist would perform to manually synthesize a compound. The feature here is that only one reaction sequence is used and the differences in the library members arise from differences in the non-common portion of the reagents. A plurality of compounds can therefore be produced more quickly than if each library member required a new route to be explored and optimized for production. The disadvantage is the limit to the diversity of compounds that can be produced by a single route and the large amount of time required (relative to the combinatorial approach) for handling compound preparation and purification on an individual basis. Efficiency is only gained when the library can be prepared in batch mode (parallel processing) usually by employing automation. Therefore the frequency at which structure-activity relationships can be updated is determined by batch size (usually hundreds into thousands to offset the overheads of automating the process) and the associated cycle time for designing, preparing, purifying, registering, transporting, assaying and reporting the data for the batch (usually months).

Because of the specialized nature of high throughput chemical synthesis and high throughput biological screening there is a perceived need in pharmaceutical research and development to logically divide responsibilities by discipline in order to maintain core competences and develop expertise in a collegiate fashion. Because of their large capital requirement, there is also a need to provide these services through one or very few “centralized” facilities. There is a perceived need also to physically divide activities on the basis of their different resource demands. For example, a chemistry department, its accommodation and equipment, is quite different from a department conducting biological research, and each differs from an information technology department. This practice of division of responsibilities coupled with high throughput technology which relies on adherence to batch processing in accordance with standard operating procedures, frequently has a potentially adverse effect: the different departments become inflexible enterprises in their own right with their own goals and the bigger they become, the more disconnected they become from other enterprises essential to the task of drug discovery. Disconnection can be both physical and temporal. Large batches of compounds from a chemistry center can end up being transported large distances to a screening center and the relative scheduling of the preparation and screening events of batched compounds is sub-optimal in respect of maximizing the use of biological data to inform compound design. Thus whilst high throughput chemistry groups may achieve high productivity in terms of compounds produced per chemist, and high throughput screening groups achieve a high number of assays run per staff member, there generally is less real-time interaction and feedback between these two activity silos than usually can be found between adjacent groups in full communication, performing these missions manually and at low throughput. However, low throughput groups incur a time and cost penalty on the enterprise focused on generating new drugs. Thus, current large pharmaceutical research and development in practice seeks a balance set towards high throughput technologies for the early lead discovery stages and graded to low throughput synthesis and assay as lead optimization approaches a clinical candidate.

Apart from the organizational difficulties, reduced interaction and feedback also arises from the nature of current high throughput methods which are based on the numerical efficiencies derived from working in very large batches in a parallel manner. Thus parallel synthesis in chemistry requires the validation of only one reaction route to prepare many compounds which will often share common structure to a substantial degree. In high throughput screening the time taken to validate the high throughput assay is recouped by its repeated high speed use across many plates of compounds. Unfortunately, this dependence on large batches to deliver numerical efficiency provides no opportunity for iterative improvement against the criteria set for a successful drug candidate. An additional barrier is set by processes designed to deal with the practical reality that synthesized and/or assayed compounds have to be physically moved from the site of preparation to the site of assay. These include isolating solid single materials, bottling, labeling, registering, storing, retrieving from store, dispensing, re-dissolving, and distributing. These processes require that sufficient amounts of compounds are prepared to allow these processes to be physically possible. Often several hundred milligrams are required to satisfy the storage and retrieval demands and transmission wastage, yet many modern assays require no more than a few thousand molecules. Indeed there is much extra to be gained in information content from assays conducted on a ‘single molecule’ scale as there is a clarity with regard to signal source, there is evidence of mechanism and the information is not obscured by aphasic information from a plurality of molecules at different stages of action. In addition, there are inconvenient waiting times involved in many of these processes, particularly if the chemistry, screening, and compound management groups are physically remote.

Other methods known in the art include manual or semi-manual chemical reaction optimization as it is routinely practiced. Manual or semi-manual iterative medicinal chemistry requires human intervention, and is very slow. For efficiency in respect of time or cost, the process is usually performed through the construction of combinatorial libraries or parallel synthesized arrays conducted in wells or flasks following a pre-conceived experimental protocol designed to test the influence of pre-decided parameters exemplified through sets of compounds in which the strength of the parameter is varied. Manual or semi-manual iterative medicinal chemistry usually requires substantial human intervention but is a very slow process involving the activities of several different knowledge disciplines which may be located at significant distances from one other. However, it should be noted that stepwise iteration using the accumulating data to inform the design of the next single compound, represents the most powerful search method and demands the fewest chemical examples to explore the greatest amount of chemical diversity space.

Another commonly practiced method known in the art is the sequential use of automated high throughput chemistry and automated high throughput screening. In automated high throughput chemistry a plurality of compounds with a familial relationship is prepared according to a standard method and placed in a compound store. In automated high throughput screening a plurality of diverse compounds drawn from a compound store is screened against a single biological target by a standardized method. In the de novo lead iteration of the present invention, by contrast, the products of a single reaction are assayed directly as single entities in one or more assays to gain information. The information is used to predict the structure of a subsequent compound with improved properties which need not have a familial relationship with the original compound nor be created through a cognate synthetic sequence.

The medicinal chemistry platform as deployed in the pharmaceutical industry is a virtual paradigm and encompasses work carried out by different disciplines usually located in different locations where the separation of the physical activities of chemical compound creation and biological assay are carried out at locations separated by more than 3 meters and separated by at least one wall.

“Originally a scientific curiosity of physicists and chemists, microfluidics now appears ready to transform traditional assay systems in academia and biotech as well as in big pharma and hospitals, with devices labeled as ‘pinhead Petri dishes’ and ‘Lab-on-a-chip’.” Clayton, Nature Methods 2, 621-627 (2005). Microfluidic devices have been known in the art for only a few years, beginning primarily with such lab-on-a-chip devices that require samples to be introduced into the device in a highly specific form, such as premixed in a homogenous reagent mixture. A review in 2003 concluded that while many microfluidic devices were in active development, integration of all laboratory functions on a chip, though the commercialization of truly hand-held, easy to use microfluidic instruments has yet to be fulfilled. Weigl, Advanced Drug Delivery Reviews, 55 (2003) 349-377, specifically incorporated herein by reference in its entirety. See also Fletcher et al., Tetrahedron 58 (2002) 4735-4757. However, advances in microfluidics have brought the integration of microfluidic and electronic components, as for example disclosed in U.S. Pat. No. 6,632,400.

Additional discussion of microfluidic chemistry may be found in Fletcher et al., Lab Chip (2002) 2:102-112; Fletcher et al., Lab on a Chip (2001) 1:115-121; Watts et al., Chem. Soc. Rev. (2005) 34:235-246; Broadwell et al., Lab on a Chip (2001) 1:66-71; Kikutani et al., Lab Chip (2002) 2:188-192; Skelton et al., Analyst (2001) 126:11-13; Haswell et al., Chem. Commun. (2001) 391-398; Wong Hawkes et al., QSAR Comb. Sci (2005) 24:712-721.

U.S. Pat. No. 6,391,622 discloses integrated systems performing a wide variety of assays and other fluid operations on a micro scale.

International Patent Application Pub. WO 2004/089533 discloses microfluidic systems.

U.S. Pat. No. 5,463,564 discloses an iterative synthesis system based on directed diversity chemical libraries.

None of the existing methods in the art provide an iterative de novo synthesis system unimpaired by the restrictions imposed by requirements for chemical structural similarity in the optimization of lead compounds. The art, therefore, is in need of improved methods and systems for drug discovery. In addition, none of the existing art shows how chemistry preparation can be operated using quantities below the level storable by normal compound management systems which directly deliver adequate quantities of pure, trackable and identifiable molecules into an assay system. In addition, none of the existing art shows how the data from the assay system can be used iteratively in an automated self-educating synthesize-assay-redesign cycling protocol to provide a “one by one” succession of new and diverse candidates for synthesis in the next cycle. In addition, none of the existing art explains how diversely structured compounds can be ranked using a common measure that provides a practical method for automated compound redesign and class-hopping (i.e. an unprejudiced leap from one active distinct chemotype to another distinct active chemotype). In addition, none of the existing art provides a basis for deriving potential synthetic methods and handling the trial thereof based on a relationship between reagent and conditions and biological outcome.

### SUMMARY OF THE INVENTION

In contrast to previous methods, a product of de novo lead iteration of the present invention is a single compound resulting from the iterative process, designed and synthesized according to the invention, avoiding the multiplicity of compounds inherent in these other methods.

The present invention differs from conventional retrosynthetic synthesis seeking programs that focus on relating the structure (atoms and bonds) of a single product with potential synthetic methods. In so doing, this new method is uniquely able to cope with real events such as multiple (and biologically active) products arising from the same reaction (by defining them by e.g. order of elution or molecular weight). In addition, the process of the present invention is able to proceed when compounds of unknown structure are generated by relating the reagents and reaction conditions to the pharmacophore (in this case expressed as field points). This would normally halt processing in programs dependent on knowledge of the atom and bond structure of the compound or compounds in the process. The ability to continue processing allows the system to run a routine in which the use of reagent analogues where the reactive moiety is conserved, can be used to detect the effect of these changes on mass-spectral and biological data. These results can still be used to drive for pharmacophoric improvement, help to establish the structure of the unknown compounds and contribute to the self-educating processes.

The present invention provides for automatic generation and direct testing of the products of a single reaction under flow conditions and under the control of a self-educating, self-improving algorithm directed to seek the best conditions for carrying the said reaction to maximize the range of possible products and maximize the yield of a selected product. In an automated lead discovery and optimization platform performing de novo lead iteration, the physical activities of chemical compound creation and biological assay are carried out at locations within 3 meters of each other without an intervening wall.

The present invention is directed to the computer-controlled generation of chemical entities with a prescribed set of biological, chemical and physical properties through the automated microfluidic de novo synthesis of single compounds which need have no obligatory relationship one to another in terms of familial structure, mode of synthesis or any other pre-conceived notion. Their diversity is limited only by the range of reagents available to the method and hardware. For convenience this mode of compound generation is referred to herein as “de novo iteration”. The present invention is also directed to the new chemical entities generated by employing the methods of the present invention.

The present invention provides a way in which the material transmission losses can be avoided, chemical synthesis routes can be rapidly validated through the use of minuscule amounts of intermediates, and the quantity of final product produced is more appropriately scaled to the actual assay requirements. Because of the micro-scaling of the reaction system, diffusion times are in the region of a few seconds and most reactions reach equilibrium or maximal product yields within the range of seconds to minutes. Reading times are similar for assays involving isolated enzymes or receptors that do not require long incubation (e.g. as with cell based assays). In addition, there is no dispensing wastage as happens with plate assays due to the need to provide a working volume in a well. In a flow assay, only the amounts of biological reagents and proteins actually used in the assay need be dispensed, and these amounts are usually at least 1000-fold less than that used in 1538 plate microtiter-plate assays.

The present invention provides a way of greatly reducing the wastage of materials and time that accrue under the present systems of lead discovery and optimization. Long cycle times arise during reaction and route validation due to the need to repeat the synthesis of potential intermediates in a relay fashion to maintain a supply in experiments. By reducing scale and integrating equipment to provide a closed loop optimization system with minimal or zero losses, reactions proceed faster and time and materials normally lost in moving materials between processes and departments are eliminated. Effectively, the present invention reduces route finding and reaction optimization losses, and the losses associated with purification, analysis and storage allowing for the consumption of only minuscule amounts of material, and yielding the information required to permit only the optimal reactions and routes to be conducted on a macro scale (if substantial amounts of material are required for such later stage processes).

One benefit of the present invention may be seen from its use of microfluidics to conduct all necessary chemistry as well as biological assays. If an assay is directly connected to a microfluidic chemistry generator, there is never a need to scale up beyond microgram levels until a compound with a satisfactory biological profile is identified. To achieve this, the realization system components must be located with some degree of proximity, although the electronic control of the automation of the Realization System permits aspects of its management to be controlled and upgraded from a remote position via a network, such as an internal network or the internet, thereby ensuring that the most relevant core competences can be brought to bear. It is also not necessary that the automation control functions and informatics and decision-making functions be co-located, providing network contact can be maintained. For example the process could be seeded on a computer in a molecular modeling department where the self-development of the algorithms could also be monitored. Meanwhile, a chemistry/biology (technology, realization?) department, possibly not even in the same country, can monitor the usage of materials from the chemical and biological reagents cassettes and optimize the contents appropriate to the dynamically changing usage resulting from self-education.

The iterative process of choosing molecules to synthesize, synthesizing them and then assaying them requires a rapid method of choosing which molecule should next be synthesized. While this function could conceptually be performed by a chemist, in practice, if the synthesis loop proceeds swiftly, a computer algorithm will be required to assess the fitness of candidate molecules with regards to criteria such as novelty, likelihood of activity, synthesizability and pharmacokinetic properties, and then to select a compound for synthesis based on these fitness scores.

The computer algorithm must be able to determine a likely activity value for a candidate molecule against a given biological target. For optimal results from the iterative process, this algorithm needs to take into account the results of each biological assay being performed in a timely fashion. The algorithm should ideally also be able to give a reasonable idea of fitness across a wide range of chemistry space, rather than being restricted to a small range of chemical structures.

According to the present invention, the computer algorithm can be divided into three parts: a pharmacophore builder, a pharmacophore fitter, and a candidate chooser. The pharmacophore builder process takes as its input one or more active molecules (where ‘active’ is defined as having a particular biological effect) in specific 3D conformations, together with a set of zero or more active and inactive molecules about which nothing need be known save their activity or inactivity, and produces a model which is capable of scoring new molecules according to both their probability of activity and to their usefulness in extending the applicability of the model.

The pharmacophore fitter process takes a specified candidate molecule, fits it to the pharmacophore produced by the pharmacophore builder process, and produces one or more scores relating to how well the candidate molecule fits the pharmacophore and how much information content that candidate molecule could add to the model. For convenience, the combination of pharmacophore builder and pharmacophore fitter processes shall be referred to herein as the ‘Autonomous System for Activity Prediction’ or ‘ASAP’ (also known as ‘Real Time Predictive Chemistry’ or RTPC).’.

The candidate chooser process chooses a compound to synthesize based on the scores produced by the ASAP system, possibly in conjunction with other scores such as synthesizability, physicochemical properties, measured or estimated pharmacokinetic properties and the like.

One way in which the fitness of molecules in structure-activity relationships can be assessed is by the ability of a compound to fulfill pharmacophoric requirements exemplified by, but not restricted to, field points as described in J. Chem. Inf. Model. 2006, 46, 665-676. The pharmacophoric field point parameters are derived from an analysis of properties in the locality of the atoms of a molecule when in its bioactive conformation and therefore reflect the environment of a binding site of a protein target. The pharmacophore field point arrangement may only have meaning within this environment. The properties it reflects may be given names for convenience, but the name given may reflect a predominant property. These measures of fitness are distinct from conventional ball and stick representations of molecules, which are a graph of the connectivity of atoms within the molecule, but which do not overtly describe physical or biological properties, nor do they define the bounds of the molecule as may be “sensed” by a binding protein. These measures of fitness are also distinct from ab initio calculated molecular orbital depictions of a molecule, as these represent the molecule in the environment of a vacuum. These measures of fitness are also distinct from models that may be notionally derived by the close approach or actual bonding of atoms within a small molecule and atoms within a protein as may be derived from graphs derived by spectroscopy techniques such as X-ray crystallography, NMR spectroscopy, and the like.

According to the present invention, the pharmacophoric entity defined by field points need not be the same as a pharmacophore as typically described in the art. The generally accepted understanding of a pharmacophore is a 3D-graph of physical and chemical properties in the regions defined by post hoc analysis of structure activity relationships of known ligands with the protein whose pharmacophore is displayed. See, e.g., published international application WO 04/023349.

For the purposes of the current invention the term “pharmacophore” has a broader meaning and is taken to mean any method providing a map from chemical structure to activity at a target protein. In addition, it may also contain elements which describe how the same compound can bind or avoid binding at other proteins. As such the use of the term pharmacophore within this invention description includes the case of a holistic representation of how a compound might display activity at the target protein but also displays information describing a variety of parameters, such as how a molecule might achieve selectivity or joint activity, bioavailability through various barriers or the lack thereof, and properties associated with toxicology, or metabolism or disposal of the said compound.

According to the present invention, the comparisons of a molecule's ability to satisfy the chemical identity, chemical purity, and biological activity in a chosen biological assay or assays against the criteria for gauging the molecule's fitness as a drug entity are based on information that is passed within a closed loop. That is not the same as passing information between the separate processes in virtual drug discovery platforms as practiced in the pharmaceutical industry where the components may be separated by more than 3 meters. In general, the pharmaceutical industry's prevalent virtual drug discovery platform requires that for the purposes of interpretation, data transferred between components must be in accordance with pre-set standards. Often these are universally accepted standards of, for example, but without restriction: IC50, EC50, Ki, Kd, or some other expression of half maximal activation or inhibitory activity or some other reliably comparable point, for example one third maximal activity. For further reproducibility and standardization these values are also often related to a standardized environment, for example, physiological conditions of temperature, pH, oxygen partial pressure, etc. Within a closed loop system of the present invention, the specifications of the measurements and the conditions under which the determinations of fitness are assessed do not require prior notification and can instead be transmitted with the data. Furthermore, within a closed loop system of the present invention, the specifications and conditions of determination can be continuously varied, and the invention will still prove useful such that the passed information can still be used to optimize the molecular properties. This feature can provide improved speed, facility, and objective determination for the property search. For example, the temperature of an assay could be raised to reduce the time taken to conduct an assay provided that this does not change the relative ranking of the molecules displaying the assayed property. Furthermore, for an assay conducted within a flowing system, the change of assay determination time resulting from the change in experimental temperature can be used to adjust the position in the flow channel where reliable readings may be taken and thus contribute to a self-optimizing, self-adjusting feature for the assay system. Examples include, but are not restricted to:

a) achieving the centering of a sigmoid saturation curve within the same given flow length of a flow assay system such that the ligand concentration required for half maximal activity may be read for a variety of assays with different incubation or determination times.

b) identifying a reading position which is not in a region of dynamic change due to the presence of unstable intermediate material, but which provides a reading of the final equilibrium condition.

c) identifying a reading position as a means of investigating or recording mechanistic aspects of the experiment assuming the reading position is in a region of dynamic change due to the presence of unstable intermediate materials. Moreover, this self-optimizing, self-adjusting feature may accomplish any combination of these identifications simultaneously.

Those with skill in the art will readily appreciate the variety of applications suitable for treatment by the methods of the present invention. These and other features of the invention are exemplified and further described in the Detailed Description of the Invention below.

In one aspect, then, the invention is directed to a method of de novo iterative synthesis, comprising the steps of:
a) selecting a candidate compound having desired pharmacophoric fit with a seed structure;
b) synthesizing the candidate compound;
c) assaying the synthesized compound, and comparing the synthesized compound to the seed structure to determine whether the synthesized compound has synthetically desirable properties, wherein if the synthesized compound does not have synthetically desirable properties, step a) is repeated for a new candidate compound, and if the synthesized compound does have synthetically desirable properties, then step d) is performed; and
d) iterating steps a) through c) wherein the synthesized compound is used as the seed structure of step a), until exhaustion.

In another aspect, the candidate compound is selected based on additional criteria selected from the group consisting of availability of reagents and viability of synthetic routes. The pharmacophoric fit may be assessed using field scoring.

In another aspect, the synthetically desirable properties comprise data selected from the group consisting of demonstrating greater bioactivity, and providing information regarding allowed, disallowed, or novel regions of space.

In yet another aspect, exhaustion occurs when subsequent synthesized compounds are not superior to previous synthesized compounds for at least 3 iterations In one aspect, step c), assaying and comparing, may be performed using ASAP.

In another aspect, the invention provides a system for de novo iterative synthesis, comprising
a) a reaction design system; and
b) a realization system;
wherein the reaction design system and realization system are capable of passing information bilaterally via computer interaction to achieve de novo iterative synthesis.

The reaction design system may comprise:
a) a database of field mapped compounds;
b) a structure fitting pharmacophore generator;
c) a computer and software; and
d) optionally, a fragment database.

Additionally, the reaction design system may be used to generate of list of candidate compounds selected from the database of field mapped compounds, and outputs instructions to the realization system to synthesize a particular candidate compound.

In one aspect, the realization system comprises
a) a microfluidic apparatus comprising a pump, reagents, reagent ports, a reaction zone, a plurality of sensors, a flow conditioner, and a flow assay system;
b) a computer and software;
wherein the realization system receives input from the reaction design system instructing the synthesis of a candidate compound, and wherein the realization system is capable of synthesizing and assaying the candidate compound.

The invention also is directed to molecules synthesized by the methods and systems of the invention.

### BRIEF DESCRIPTION OF THE DRAWINGS

FIG. 1 is a schematic diagram illustrating the general sequence of events in one embodiment of the method of the invention.

FIG. 2 is a schematic diagram illustrating an embodiment of part of the Reaction Design System of the present invention which carries out the function of a Pharmacophore Generator. Together with FIG. 3 this constitutes the Reaction Design System.

FIG. 3 is a schematic diagram illustrating an embodiment of part of the Reaction Design System of the present invention which carries out the function of a Candidate Chooser in the De Novo Design Process. Together with FIG. 2 this constitutes the Reaction Design System

FIG. 4 is a schematic diagram illustrating an embodiment of the Realization System of the present invention, including all parts of the system.

FIG. 5 is a schematic illustration of a chemistry generator system.

FIG. 6 is a schematic illustration of an Analytical and separation system.

FIG. 7 is a schematic illustration of an Assay system

FIG. 8 provides molecular structures used in the ASAP process of Example 3(a).

FIG. 9 provides results from the ASAP process further described in Example 3(b).

### DETAILED DESCRIPTION OF THE INVENTION

In accordance with the present invention, the fitness of molecules in structure-activity relationships is assessed by the ability of a compound to fulfill pharmacophoric requirements exemplified by, but not restricted to, field points. The de novo iteration method of the invention generally comprises a series of loops, as described in greater detail below.

Iterative Loops Incorporating Reaction Design and Realization Systems

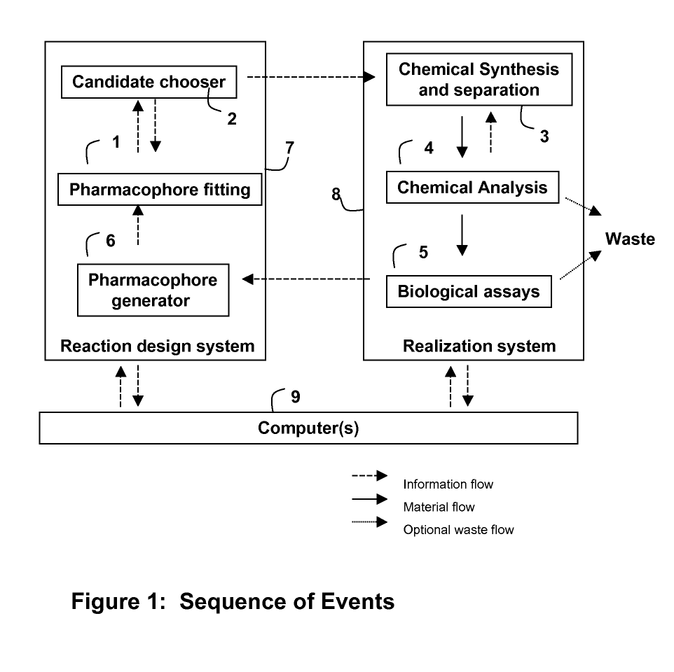

As depicted schematically in FIG. 1 and with reference thereto, the methods of the present invention are conducted in a logical system consisting of three loops: an overall loop (1→2→3→4→5→6→1 etc.) that designs and performs a chemical reaction and assesses the products using biological parameters; an iterative compound design loop (1→2→1 etc) which forms part of the Reaction Design System (7) and a chemistry testing loop (3→4→3 etc) which forms part of the Realization System (8). The purpose of each of these loops is to present hypotheses to be tested practically in the hardware system (8). The Reaction Design and Realization systems (7,8) are bidirectionally coupled to a computer or computers (9) to supply data to the computers and provide control signals to the Realization System and the Reaction Design System. The data generated by progress through these systems is then stored, and the success or failure of the postulates is used as the basis of a self-educating system which, through successive iterations, drives towards synthesis of compounds which meet a set of pre-set criteria for an enhanced biological profile. The iterative process begins by seeding the Pharmacophore Generator (6) with information to build an initial pharmacophore (for example, a single active molecule), and terminates when a desired goal is reached or the process is deliberately abandoned.

The Pharmacophore Fitting (1) and Candidate Chooser (2) subroutines identify a series of compounds predicted to fit the activity model by either seeking a suitable template from a large diverse virtual structure database or by trial assembly of molecular fragments and then selects one compound for synthesis based on weighted factors such as the scores obtained with the activity model, the availability of reagents and the perceived viability of synthetic routes. The reagents required for the synthesis over one or several steps are then defined and the task is handed to the hardware control system to perform the actions necessary to test and optimize the synthesis pathway.

The data from the chemistry assays are analyzed and conditions re-set by goal-seeking routines operating within the loop (3→4→3) etc. designed to target the highest yield of the product defined by the decision-making software (7). When preset criteria are reached, this product and/or other similar products are separated in a stream by chromatography (or other applicable and available resolution techniques) and approximate concentrations are determined. The products are identified according to several criteria: (i) that they are products and not reagents, (ii) their provenance as a product of a recorded set of reagents under a recorded set of reaction conditions (and the structure that might predict), (iii) their position relative to other compounds in the chromatographic eluate and (iv) analytical data such as might be obtained from mass spectrometry (e.g. molecular weight) or chemiluminescent nitrogen detection (proportion of nitrogen content). The separated products are then singly diluted and submitted to the biological assays (5).

The data from the biological assays are provided to the Pharmacophore Generator (6) and may be used to update the pharmacophore. The updated pharmacophore is then used in subsequent Pharmacophore Fitting (1) steps. In this way the system as a whole learns from the data that it is producing.

The process continues in an iterative and cyclic fashion until the pharmacophore is as fully defined as possible or the desired level of activity of the last synthesized molecule has been reached. Contingency routes for failure, restart, redesign or chemotype hopping are provided by a field-based virtual screening tool and a molecule builder based on the assembly of the field patterns of molecular fragments. All tools and processes use molecular field pattern comparisons and are free from the constraints of molecular structure.

Each overall loop of the process may be considered to be the testing of a hypothesis, namely, that the mixture of reagents under the particular extant conditions will produce a compound that has an improved biological activity profile. The results of the hypothesis test are sharply divided into ‘true’ and ‘false’ for purposes of processing. At one extreme of behavior this will provide through the chemistry optimizing loop (3→4→3 etc.) a single compound supported by analytical data to probably be the improved structure predicted by the structure fitting sub-routine (3, FIG. 1).

At another extreme there may be several products none of which return analytical data to support the structure predicted by the structure fitting sub-routine (3). In this case two actions are taken: The first is to apply a selection filter based on notions of what constitutes drug-like structure, such as those principles espoused by Lipinski and others/In 1997 Christopher Lipinski published a seminal paper identifying a series of features commonly found in orally active drugs. These features are referred to as Lipinski's rule of five and can be used as a rule of thumb to indicate whether a molecule is likely to be orally bioavailable (bioactive). The “rule of five” is so called because most of the features start with the number five. In general, an orally active drug has:
not more than 5 hydrogen bond donors (OH and NH groups)
not more than 10 hydrogen bond acceptors (notably N and O)
a molecular weight under 500
a LogP under 5

CA Lipinski, Adv. Drug Del. Rev. 1997, 23, 3. The selection filter, based on the data available, may remove from any further consideration any compounds that lie well away from the criteria for drug-like behavior; for example, a molecular weight as detected by mass spectrometry as being well in excess of 500 Da or lipophilicity as judged by chromatographic retention times that lies well outside the range of logP values 2 to 5 and generally accepted to be required for cell penetration and bioavailability. However, it is accepted that in certain cases current teaching would be to admit examples exceeding the limits by a small degree and in certain areas of work (e.g. antibacterial or neurological targets) it may be desirable to exemplify enhanced polarity or lipophilicity in compound test sets. The second is to test all compounds which pass the first filter for biological activity and to relate the biological data to the compound information regarding provenance, retention time, and analytical data as described above. If any of the compounds is sufficiently significant in meeting the criteria for the desired biological profile to vie with the predicted series, then in the manner of conventional investigative chemistry, a variety of modified reagents are set to react such that by collecting the analytical data over the subsequent iterations of the loops, the contributions of reagents can be evaluated and the structure of the unknown product can be elucidated.

In this process the reagents are chosen not only to assist identification, but also on the basis of assembling an improved molecule based on the assembly of fractional pharmacophores for reagent-based fragments. This process can have several outcomes: (i) the structure of a new chemical class capable of delivering an enhanced biological profile will be identified; (ii) the process will fail to elucidate structure but enhanced biological properties are generated; (iii) the structure of a new chemical class is identified but it is unsuccessful in competition with other classes for delivering an enhanced profile and is eliminated from the search; and (iv) the process is unsuccessful in delivering either the identity of the molecules or compounds with enhanced biological profile. In the case of outcome (ii), further pursuit will likely ultimately identify the molecule, but if the biological goal is reached before structure is elucidated, then the invention is still considered to have achieved its desired result because it will be possible to identify the compounds using off-line techniques such as X-ray crystallography.

Thus, in one embodiment, the present invention provides a system comprising two sub-systems: a Reaction Design System (7) (alternatively referred to herein as “Compound Design System”) and a Realization System (8). Each system is controlled by a computer or computers and each is capable of autonomous decisions. In addition the systems can jointly transact decisions and actions by the bilateral passage of information and requests. The same physical computer can serve both functions, or several computers can be networked to provide the required functionality.

To best provide the efficiency conferred by use of the present invention, it is preferred that the components of the Realization System (8) are small and contained together in a close-coupled fashion to minimize the physical process flow length, for example, by employing microfluidics technology to carry out all chemistry. The processes within the Reaction Design System are electronic and the locations at which individual subroutines are run have no influence on process efficiency, provided that they are efficiently electronically networked. The Realization system does not need to be co-located with the Reaction Design System or any component thereof. The computer or computers (9) may be severally located independently of each other and of the components of the Compound Design System (7) and the Realization System (8).

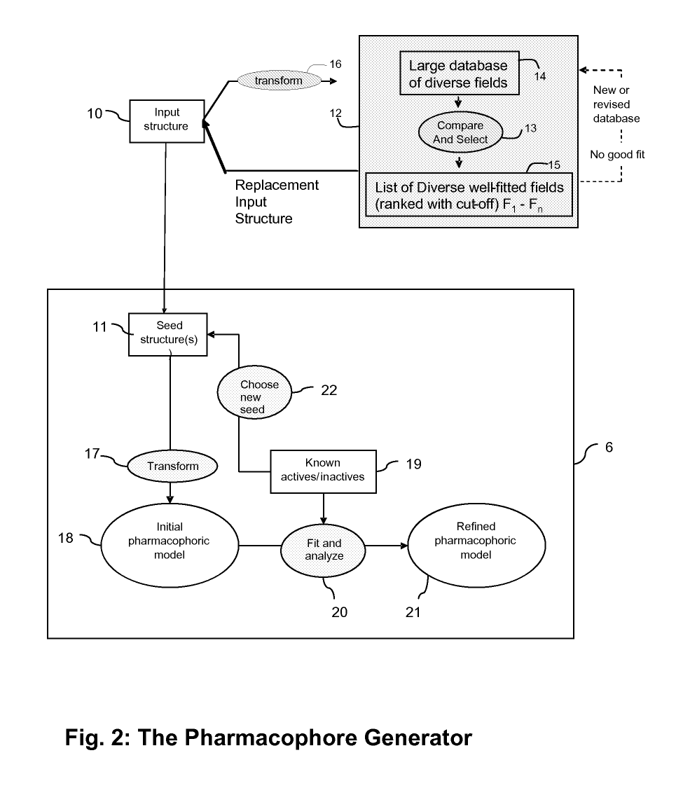

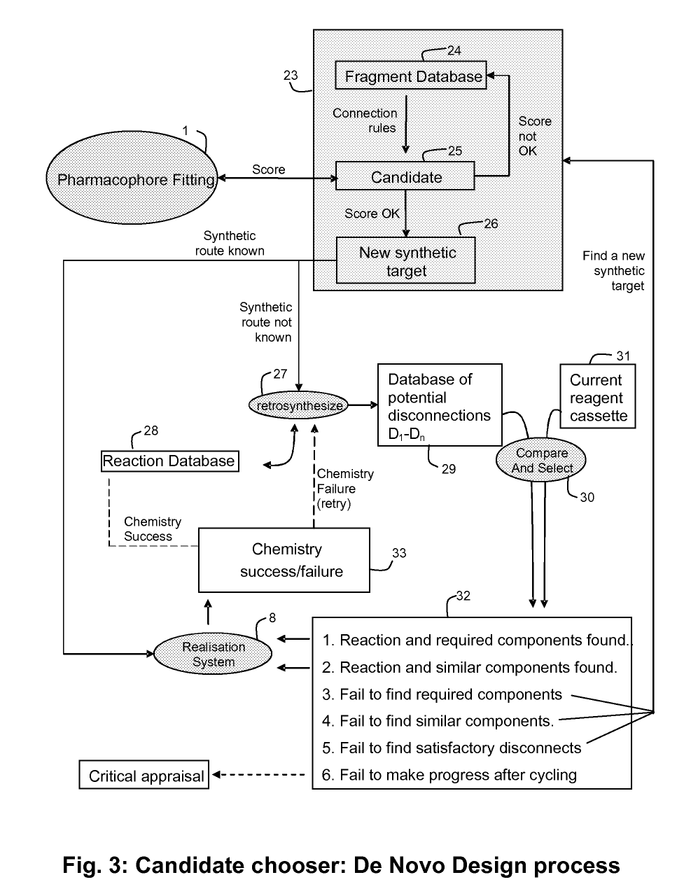

The Reaction Design System (7) comprises a set of computational programs which in total perform all the actions as described in FIG. 1, namely pharmacophore generation (6), pharmacophore fitting (1), and choosing a synthesis candidate and reaction pathway(2). The reaction identified is that reaction predicted to produce a product with an enhanced biological activity profile relative to the input or seed compound, or alternatively, in subsequent iterations of the loops, a product prepared in a previous iteration. This prediction is tested by attempting the reaction and testing selected products in the Realization System (8). The actions of FIG. 1 are achieved through the routines and components depicted schematically in FIGS. 2 and 3 which together constitute the Reaction Design System.

The Reaction Design System

The components and technologies involved in the Reaction Design System are specified in detail below.

Pharmacophores and Field Points

While several methods are available to those of skill in the art, such as structurally-derived pharmacophores as used in the Catalyst software from Accelrys, or docking to protein structures using software such as Gold from CCDC, one suitable method for defining pharmacophores is that described in US Patent Application Pub. No. 2006/0129323 entitled “Comparison of Molecules Using Field Points” (see also published international patent application WO 04023349), incorporated herein in their entirety. This method is referred to herein as “field scoring”.

Field scoring involves defining one or more fields around a given 3D conformation of a molecule, determining the position and value of ‘field points’ based on these fields, and then using both the field points and the actual fields of two molecules to provide a similarity score for those molecules.

In order to calculate field points, a field definition must be adopted. One known field definition for molecular mechanical models uses positive and negative electrostatic interaction fields in combination with a surface interaction field. The two electrostatic interaction fields are defined by the interaction energy of a specific charged ‘probe’ molecule with the molecule of interest. For example, a probe the size of an oxygen atom, with either a +1 or a −1 elemental charge, can be used. The field value at a given point is the interaction energy of the molecule with the probe atom sited with its centre at that point. The surface interaction field is defined by the van der Waals interaction energy of a neutral ‘probe’ with the molecule, for example an uncharged oxygen atom.

Other field definitions have been used, for example ones that include electrostatic fields calculated from quantum molecular methods, and ones that include hydrophobic fields calculated from the electrostatic field and its partial derivatives. In principle, any field definition can be used provided that its value can be defined at any point in space around the molecule.

Once the field definition has been made, the field points of the molecule need to be calculated. With the molecular modeling approach, the field points are subdivided into a number of subsets, one for each field type, with each subset being calculated separately. The field points for a molecule are the values and locations of the extrema of its field, i.e. maxima and/or minima. The final set of field points from each field type can be filtered to remove duplicate extrema and small extrema if desired. The field point set encodes a large amount of information about the properties of the molecule, especially regarding its interaction with other molecules. The electrostatic field points encode information about the preferred hydrogen-bonding and electronic charge environment of the molecule, while the surface interaction field points encode the molecule's steric bulk.

The basic assumption underlying the field point approach is that two molecules which have similar sets of field points should have similar interactions with other molecules and hence should have similar biological activities. In other words, if molecule A has a certain biological activity, and molecule B is calculated to be similar to molecule A in a relevant conformation, then it is concluded that molecule B potentially has the same biological activity. With the field point approach, the similarity between conformations of two molecules in a particular alignment is calculated according to a scoring formula which depends on the positions and values of the field points and the values of the fields sampled at a number of points around each molecule. In particular, the scoring formula for a given alignment may depend on the values of the field points for the first molecule and the values of the fields for the second molecule at positions corresponding to the positions of the field points of the first molecule, and vice versa. The result of the formula, i.e. the score, is a scalar quantity referred to as the field similarity value. The act of comparing fields from two molecules is sometimes referred to as field alignment, field overlay or a field overlay process by virtue of the fact that the calculation of the field similarity value requires an alignment of the two molecules.

By way of example, suppose that molecules A and B are to be compared for similarity. Molecule A is known to bind to a particular protein. The conformation of A when bound to that protein is also known. Molecule B is a new candidate molecule for potentially binding to the same protein. To carry out the comparison calculation, the bound conformation of A is compared to multiple conformations of B. Multiple conformations of B are tried, since, if B is able to bind to the protein, the conformation of B which allows such binding is not yet known. For each conformation of B, the alignment of that conformation which maximizes the field similarity to A is located, for example by a Monte Carlo search algorithm. The best such similarity score over all conformations of B is taken as the overall field similarity score between A and B.

In another example, the bound conformation of molecule A may not be known, even though it is known that molecule A binds to a particular protein. In that case, the comparison process will compare multiple conformations of A successively with multiple conformations of B, at each stage locating the alignment with the maximal field similarity score, and taking the highest such score as the overall field similarity score between A and B.

The details of one implementation of field similarity scoring are described in Cheeseright, T. et al, . J. Chem. Inf Model. 2006, 46:665-676.

Pharmacophore Generation (6)

A preferred embodiment of a pharmacophore in the ASAP system consists of the fields and field point patterns of a set of one or more ‘seed molecules’, combined with a representation of ‘allowed’ and ‘disallowed’ regions of space around these molecules (as determined on a grid, for example). Each field point may also be optionally labeled with an ‘importance’ value which is a numerical value controlling the weight that that field point has in assessing the field similarity of a candidate molecule to the pharmacophore.

The process of generating such a pharmacophore process is show in FIG. 2. The chemical structure of an input molecule (10) that has shown evidence of displaying desired biological properties is obtained and represented in the commonplace depiction of atom connectivity by showing the formal bonds that exist between atoms at room temperature. A 3D structure (conformation) which is close to the bioactive conformation should ideally be obtained for the input molecule. This conformation may be obtained by experimental means (e.g. x-ray crystallography or NMR), by computer modeling (e.g. Catalyst or FieldTemplater), by educated guesswork by an experienced chemist or modeler, or otherwise.

The input structure may be promoted in two ways; a) by designating it as the seed structure directly (11) or b) if some of its properties are deemed poor in potency, solubility or other important attribute, it may be used to search in a large virtual screen (12) of field patterns for other commercial or proprietary diverse compounds having a high probability of action at the desired target using procedures (13), (14) and (15). This and all subsequent processes use unique field descriptors rather than structural data to compare and fit new chemical entities.

The field properties of the entire ensemble of individual atoms (exemplified by but not restricted to electronegativity, softness, nucleophilicity, hardness, electrophilicity, and the like) of a seeding molecule are transformed using a software routine (16) to produce a representation of its chemical and biological properties as presented near to or within the surface of the molecule (as defined by the blended orbital limits defined by a 95% probability of finding an electron) and as appreciated by a binding site in a protein in which the molecule is likely to show a dissociation constant of 10⁻³-10⁻¹⁰. These molecular descriptions are then codified into a new set of data that may be more easily handled in computational assessments of molecular fitness. While several methods are available to those of skill in the art, one such suitable method is, as discussed above, described in US Patent Application Pub. No. 2006/0129323 entitled “Comparison of Molecules Using Field Points” (see also published international patent application WO 04/023349) each of which is hereby incorporated in their entirety. The terms ‘field points’, field point pharmacophore and pharmacophore have more general meanings in this current application to cover the products of other but similar routines to those described in US Patent Application Pub. No. 2006/0129323.

The field point template (field pattern) obtained by transforming the atom connectivity structure is then submitted to a software program (13) designed to compare it with the field patterns contained in a large database (14) of diverse field patterns representing diverse small molecules. The comparison routine aims to find molecules that would have an appreciable ability to display drug-like behavior as predicted by the similarity of their field patterns to the input field point template and weighted or filtered using protein coordinate data (if available) and any of the well-known and accepted methods (e.g. Lipinski et al. supra) in making comparisons and selections. The output of this search is a set of molecular structures ranked by the similarity of fitted fields from which the best n (where n is typically about 200, but may range from about 100 to about 3000) are held as a potential list (15) of new and diverse input or seed structures (designated F1-Fn, where F1 is the best fitted field). The retained templates in the list (15) demonstrate sufficient 3D-overlay of physical and chemical properties with the search template as judged, for example, by closeness (similarity) of alignment of the property centre positions and strengths of the field components relative to the seed compound based on Simplex optimization of all field components. Whilst the method of comparison may intuitively be based on a field point by field point comparison, this is naïve and a more rigorous and accurate method is described in Cheeseright, T. et al. J. Chem. Inf. Model. 2006, 46:665-676, incorporated herein in its entirety, without compromising computational speed.

This list of compounds (15) can then be assayed or otherwise examined to determine the candidate most likely to display the combination of biological activity and other properties that most closely matches the desired set of properties. This candidate can then be used to replace the input structure (10).

In the event of no substantial fit (as set by operating criteria) being found within the database, two resolutions may be employed; 1) other available databases can be converted to field pattern format and searched, or the current database can be extended from experience gained from the invention or from outside sources; or 2) the initial molecule (10) can be used directly as the seed (11).

The fields and field points of the seed molecule (11) are determined (17) as described earlier (see 16). This set of fields and field points is an initial pharmacophore (18), and may be used without further modification. Alternatively, the pharmacophore can then be refined and extended by considering one or more ‘training set’ compounds (19). The ‘training set’ is a set of molecules for which one or more relevant biological properties have been measured.

For each such training set structure, a set of conformations of that structure are generated. Multiple algorithms are known for this purpose to those skilled in the art. Each of the set of conformations is fitted to the initial pharmacophoric model and a score obtained (by the method outlined in J. Chem. Inf. Model. 2006, 46, 665-676) (20). The set of highest-scoring fitted conformations is kept, and a weighting factor is assigned to each alignment according to the score of that alignment, the score of the best-scoring alignment for that structure, and the number of alignments found which have higher scores. The purpose of the weighting system is to assess the confidence that can be placed in each alignment: if a particular structure possesses only one high-scoring alignment to the initial model, with all other conformations and alignments having a significantly lower score, then the confidence (and hence the weight) assigned to that alignment will be high. If, on the other hand, a given alignment has a low overall score and there exist many alignments for that structure which have higher scores, then the confidence (and hence the weight) assigned to that alignment will be low.

Each such alignment is then examined in the context of the known biological properties of that molecule. If the molecule is active and the aligned structure protrudes beyond the extents of the seed, then the regions of space that are occupied by the aligned structure and the seed can be labeled ‘allowed’, as there is evidence that the placement of atoms in those regions does not directly impede activity. If the molecule is inactive (has an activity significantly less than that of the seed molecule(s)) and the aligned structure protrudes beyond the extents of the seed molecule(s), then the regions of space that are occupied by the aligned structure but not by the seed molecule(s) can be labeled ‘disallowed’, as there is evidence that the placement of atoms in those regions may impede activity. In each case, whether the label is applied and the extent to which that label is applied depends on the weighting factor associated with that alignment.

As a further step, regions of space around the seed molecule(s) which are neither known with high confidence to be ‘disallowed’ or known with high confidence to be ‘allowed’ may be marked as ‘unknown’.

Having used the alignments to map out ‘disallowed’, ‘allowed’ and ‘unknown’ regions around the seed, an optional further step may be taken. The field patterns around the alignments may be used in conjunction with the biological property data to ascertain which parts of the field pattern around the seed molecule(s) are more important in determining biological activity and which are less important. The field pattern around the seed can be modified accordingly so as to emphasize the important regions at the expense of the less important regions. As part of this process a confidence level can be assigned to each field feature according to how much information is available to assign its importance.

As an extension, the seed (11) may comprise several molecules with pre-determined alignments and 3D conformations. In this case, the process of aligning and fitting a structure to the seed involves aligning the structure to the seed, calculating its field similarity to all of the molecules contained in the seed separately, and then combining these similarity values into an overall similarity score against the seed as a whole. As a further extension, this combination may involve assigning a weighting factor to each molecule within the seed and utilizing these weighting factors in the calculation of the overall similarity score.

The final pharmacophore (21) obtained from the analysis of the training set comprises the initial pharmacophoric model from the seed together with the allowed, disallowed and unknown regions and the importance values assigned to each field feature.

As the pharmacophore evolves, the existing known active compounds are periodically reassessed. If any of these compounds are assessed to be superior to the original seed or seeds (in terms of biological activity, desirable physicochemical or pharmacokinetic properties or otherwise) then provided that a suitable guess as to the bound conformation of that compound can be made (by fitting to the pharmacophore or otherwise), that compound can be either used as a new seed to generate a new pharmacophore model or alternatively appended to the list of existing seeds and the new set of seeds used to generate a new pharmacophore model (22).

Pharmacophore Fitting (1)

Molecules can be tested against the pharmacophore to obtain a desirability score which is derived from two components:
1. How likely is this molecule to be active?
2. How much information do we gain from making this molecule, i.e. how novel is it?
   The process used to assess new molecules and derive these two scores is outlined below.
1. Populate the conformation space of the new molecule
2. Align and score the new molecule to the seed using the seed field pattern that is weighted by the importance of each field point. The alignment and scoring is performed over every conformation of the new molecule.
3. Apply a penalty to the activity score for each atom that enters disallowed space, weighting the penalty by the confidence that we have in this region of disallowed space.
4. Construct an informativeness score for entering 3D space that has not been previously explored; i.e. is neither allowed nor disallowed.
5. Modify the informativeness score according to how well the molecule matches field points whose importance is not well known.
6. Weight the informativeness score according to the confidence that we have in the alignment.
7. Output the penalized field similarity score (the ‘fitness’ score, representing potential activity) and the weighted informativeness score (representing how much potential information can be gained from making and testing this molecule).

This fitness score is a measure of likely activity: molecules with a high fitness score should be highly enriched in actives when compared to molecules with a low fitness score.

The informativeness score is a measure of how much the model would change if information on the new molecule's activity (or lack of it) was known. For example, a new molecule whose highest-scoring alignments all extended part of the new molecule into a region of space around the seed that had previously been designated ‘unknown’ would have a relatively high informativeness score: if the molecule turned out to be active, that region of space could be marked ‘allowed’ rather than ‘unknown’, and if not then that region could be marked ‘disallowed’ rather than ‘unknown’. In either case the model would change significantly based on the knowledge of the activity of the new molecule.

The choice of which compound to make based on these scores will depend on the aims of the iterative system. In the early stages, where many parts of the pharmacophore have only low confidence values assigned to them, priority could be given to molecules with a high informativeness score. These molecules may or may not be active, but the knowledge of their activity or the lack thereof provides valuable information which can be used to improve the pharmacophore. Once the pharmacophore is more robust, the fitness score can be emphasized more, which will result in molecules with a high probability of activity being selected for synthesis. In this way the iterative system is able to utilize the ASAP pharmacophore model to both guide the evolution of its pharmacophore and then to use that pharmacophore to select the best possible candidates for synthesis utilizing as much of the available information as possible.

Candidate Choice and Reaction Design (2)

The candidate choice and reaction design module (2) uses the pharmacophore fitting module (1) to choose a candidate molecule for synthesis by the Realization System (8). One mode of operation for the candidate choice and reaction design module (2) involves de novo compound design as shown in FIG. 3.

A de novo compound designer (23) generates new synthesis targets based on the assembly of fragmentary fields derived from structural fragments (described by their atom connectivity) contained in the fragment database (24). The fragment database is associated with a set of connection rules which specify how the fragments may be connected together. The set of fragments and connection rules may be completely general or highly specific. At the extreme of the general case, a set of single-atom fragments may be combined with connection rules allowing any fragment to be connected to any other fragment. This would allow the generation of virtually any conceivable organic molecule. A preferred and more specific scenario involves the fragment database corresponding to the set of reagents available to the Realization System, together with a set of connection rules specifying the chemical reactions available to the Realization System. Such a fragment database would allow the construction of a more restricted set of molecules but would have the advantage that any molecule constructed using that database would automatically have a viable synthetic route known.

A search algorithm within the de novo compound designer (23) searches within the chemistry space defined by the fragment database (24) and connection rules to find compounds that have a high score according to the Pharmacophore Fitting module (1). This search process could be exhaustive i.e. all possible molecules able to be generated by the fragment database and connection rules are enumerated (25) and scored. Alternatively, a stochastic search algorithm (e.g. simplex search, Monte Carlo search or a genetic algorithm) could be used to search the chemistry space examining a limited set of candidate molecules (25) and eventually selecting a high-scoring candidate for synthesis (26).

If the synthesis candidate was constructed in such a fashion that a plausible synthetic route is known, then the candidate can be submitted directly to the Realization System (8). Otherwise, the target structure is submitted to any or all of a variety of organic molecule retrosynthesis routines (27) which are publicly available. Alternatively a routine to seek all of the atomic connections in the target that can be made in accordance with any of the synthesis methods and rules contained in a reaction database (28) is run and the output is passed to a database (29) of potential disconnections (D1-Dn). Further reference to the field fragment database (24) may augment the database (29) since it may consist of fragments expected to be synthetically tractable. Another computer program (30) methodically performs disconnections in silico to identify what reagents would be necessary to achieve the connection. These are compared with a database describing the contents of the current reagent cassette (31) to assess the availability of that reagent or a similar reagent. This comparison is possible because the identity of the actual reagents present in the cassette and extensive additional information regarding the class (e.g. amine, etc.) and the properties (e.g. additional functional group; size; degree of branching; lipophilicity; etc.) are previously recorded to a data chip or a database residing either in the cassette or on a computer. If a reagent set cannot be based on one of the disconnections D₁ to D_{KNY}, then a recursive program is triggered which considers an increasing number of disconnections up to a predetermined maximum, and a synthesis strategy consisting of several steps is planned. Rules embedded in routine (30) establish the order in which the steps will be carried out, but where the program scoring system shows no strong preference for a particular order in which the steps of a system may be carried out in a different order in different realization events.

Box (32) shows various output conditions for any cycle of routine (30). If a suitable disconnection and reagent set are found to be present then an attempt will be made to realize the chemistry and (if successful) obtain biological assay data via the Realization System (8). Data are sent back from the Realization System (8) to record whether the chemistry was successful or not and (if appropriate) what results were obtained in biological assays. These outputs are shown in box (33). If the chemistry is successful this fact is registered in the reaction database by increasing the value weighting of the reaction used and storing information regarding the successful reagents. The biological data are judged according to preset criteria and scored in accordance with a desired scoring scheme, such as ‘good’, ‘bad’ or ‘unhelpful’, and returned to the ASAP pharmacophore generator (6) where the ‘good and bad’ information is used to refine and update the field and volume categories described earlier, and to suggest further modifications to test any undefined or ambiguous regions of the current molecule. If the chemistry is not successful then an alternative retrosynthetic pathway can be searched for. If no satisfactory synthetic route leading to the synthesis of the synthetic target (26) or a close analogue is found, then the failure is recorded and the de novo compound designer (23) used to find a different synthetic target.

The cycle from the de novo compound designer (23) through the Realization System is repeated, with updating of the pharmacophore model (6) as biological results are obtained, until exhaustion occurs. Overall, depending on the particular circumstances of a particular synthesis program, exhaustion of the cycling process will lead to the following conditions and actions:

a) realization of a new compound with improved properties (usually but not exclusively biological activity). Under these circumstances, the molecule can be delivered to preclinical investigation, and/or used to derive new diverse chemotypes by being chosen as a new seed structure (22).

b) failure to find any valuable improvements will be subjected to off-line critical appraisal.

The Realization System

The Realization System (8) represents a system comprised of hardware and software which in response to information passed and requests made by the Reaction Design System described above and schematically shown in FIG. 2, is capable of managing and executing at least the following tasks:

a) Retrieving specific reactants from a store containing a large variety of reagents

b) Introducing them into a microchannel flow system whilst maintaining a reasonably constant flow

c) Containing a reaction occurring in a portion of the flow system

d) Without storage, moving the reaction mixture to a separation system capable of resolving the wanted components

e) Sensing the passage of reaction output past a fixed point to estimate flow component integrity, purity, identity, consistency, concentration, or quantity

f) Removing a minute fraction of selected flow components to off-line analytical devices to estimate flow component integrity, purity, identity, consistency, concentration, or quantity

g) Without breaking flow, pass the selections of the outflow from the flow and concentration adjusting module into a device capable of conducting an assay under flow conditions

h) Without storage, delivering the wanted components to an assay system capable of carrying out a chemical or biochemical or biological assay

i) Using a computer to receive and store data from all databases, clocks, and sensing systems which constitute the hardware, formulate decisions regarding how the hardware components will operate individually or in combination to carry out the requests made, and return the information required by the decision making system described in FIG. 2.

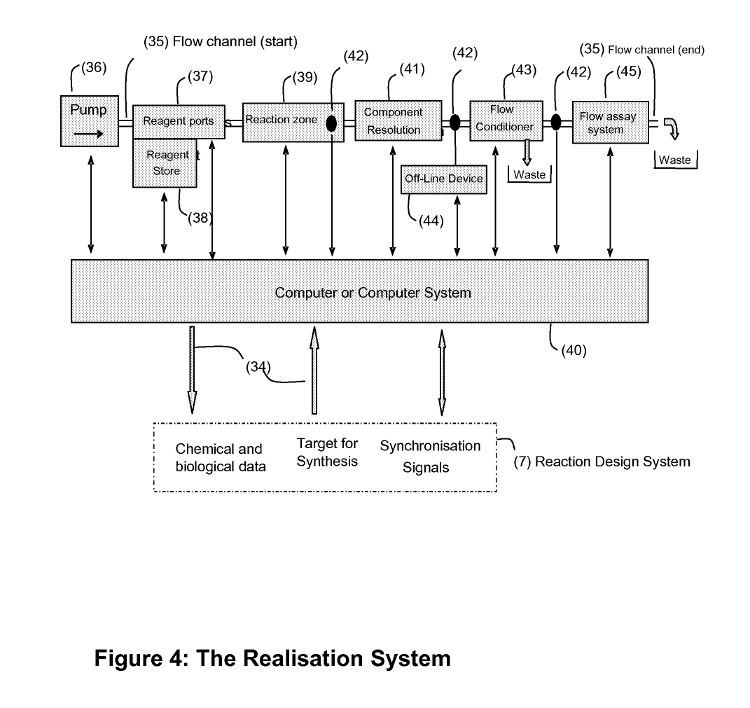

The Realization System itself is shown schematically in FIG. 4. The two block arrows (34) show the interconnection with the Reaction Design Module (7) depicted in FIG. 2. In one embodiment of the present invention, the hardware of the Realization System comprises:

a) A Flow Channel (35) with cross sectional area in the range 750 to 15,000 sq microns (usually about 50×15 u). This range can be broadened without compromise in three ways:

(1) in fusing the glass chip together there is a limit to how small or shallow a feature can be on a surface offered up for bonding to another glass surface for heat fusion bonding without it being obliterated. Gluing is a worse option because glue fills the channels and does not make a chemically resistant bond, therefore heat fusion is currently best. 2 microns probably represents the current limit but features below 10 microns are probably vulnerable in practice and failure rates are high below this figure.

(2) the reacting solutions must be pumped along a substantial length of microchannel. The dimension given requires a pump which can show nanoliter precision whilst developing a pressure of about 100 bar instant and 50 bar running.

(3) internal coating of the channel (catalysts and other coatings) may require diffusion of gas which is very slow when channels are small.

At this time, 50×15 um represents about a favored embodiment when these factors are taken in combination. It is likely that the channels could be smaller, but as of today there are no commercially available pumps. At this size the chemistry generator still supplies many orders of magnitude greater amounts of compound than an assay requires, therefore as smaller sizes become available, the invention is equally practicable at such smaller sizes. Consuming chemical reagents at this level also presents no supply chain issue. As the assay scale is set independently of the amount of chemical available (i.e. surplus compound goes to waste) there is no issue regarding the unnecessary consumption of highly rare and expensive biological reagents.

Overall, for chemistry this represents the current state of the art, however if the technology allows smaller scale (smaller channel size), the invention would be amenable to such smaller scales because the ability to control chemistry through reactor heterogeneity and surface to bulk effects (i.e. dominance of managed surface effects) would improve greatly right down to the limit of a few or single molecules where scale-related effects work more beneficially in our favor. To scale up has use but not in the “information only” mode of this invention. However, if we were to go on with the aim of producing visible and storable amounts, the issue becomes one of guessing at which point the benefits of the small system run out. If we derive benefit from working at this small scale it is because the system lacks turbulence and the surface to bulk ratio is high. Although there are theoretical calculations that can be made regarding (a) the breakdown of non-turbulent behavior (based on Reynolds Number) or stoichiometry between solution components and surface components, it has been generally found that “special effects” based on these sorts of considerations are impacted at about 1 mm. Advantages due to special and temporal effects due to flow alone (such as very low variance in thermally mediated change by passing through a hot zone (FIFO effect) of course remain unaffected. A 1 mm system (a so-called meso system) requires fast flow rates and long channels (many meters). The consumption of reagents and production of product can lie in the tens of mg/min range. These performance metrics are excellent for a production system but run against the principles of an “information only” system. Therefore, about 1 mm diameter is generally used as an upper limit, but there may be a lower limit other than the state of the fabrication and hardware art because assays already verge toward single molecule scale (at least when a good fluorescent reporter can be set).

b) A Pump (36) with an optional local control and sensor system, capable of pumping a solvent at constant flow rates in the region of 1 nL to 1 uL per minute in the direction shown by the arrow.

c) Reagent Ports (37) physically connected to a Reagent Store (38) with optional local control and sensor system through which a plurality of reagents may be added to the flow channel (35) in a controlled and adjustable manner. Pinch or loop methodology may be a suitable method for achieving introduction without materially disturbing flow rate.

d) A Reagent Store (38) physically connected to the Reagent Ports (37) with an optional local control and sensor system capable of storing a large plurality of reagents in either solid form or as solutions in a suitable solvent, or of preparing these solutions on demand, and capable of retrieving and delivering reagents to the Reagent Ports (37).

e) A Reaction Zone (39) being a portion of the flow channel in which the reagents introduced through the ports (37) will contain the reagents as they undergo reaction in flow and where the conditions of operation can be changed in response to signals from an optional local control and sensor system by means of Computer (40)

f) A Component Resolution Device (41) with an optional local control and sensor system which is capable of spatially and/or temporally resolving or partially resolving the components of the mixture passed from the Reaction Zone (39).

g) Sensors or Sensor Ports (42) which can occur anywhere in the system. They are exemplified in FIG. 4 in three modes: a sensor placed within the Reaction Zone (39); a sensor port between the Component Resolution Device (41) and The Flow Conditioner (43) and used to pass a negligible portion of the flow to an off-line device (44); and a Sensor within the Flow Channel (43) between the Flow Conditioner (48) and the Flow Assay (45).

h) A Flow Conditioner (43) with an optional local control and sensor system which can be controlled from the computer (40) to adjust flow rates, adjust flow concentrations or port materials from the Flow Channel to waste.

i) A Flow Assay Subsystem (45) with an optional local control and sensor system capable of determining biological data for separated components from the reaction and in which the conditions of operation can be changed in response to signals from the Computer (40)

j) A Computer or Interconnected Set of Computers (40) which provide overall control of the Realization System (8) and maintain synchrony with actions occurring within the Reaction Design Module (7) through signals passed between the two systems.

Those of skill in the art will appreciate the variety of hardware adaptable to accomplishing the functions of the Realization System (8).

In another embodiment of the present invention the functions of the computer within the Realization System (8) may include the following, but are not limited thereto:

a) Maintain an inventory of physical and chemical data relating to material in use in the system

b) Maintain an inventory of hardware components present in the system and their capabilities

c) Maintain an inventory of assay data—provide an electronic interface suitable for receiving and storing data from assay devices forming part of a closed loop system as described above

d) Provide a database of subroutines that can be called singly or severally and parameterized to carry out a sequence of actions

e) Provide a knowledge base for how the methods of the Reaction Database should be translated into hardware actions and sequences and take account of the needs of the different forms that reagents might take (the necessary data are encoded in the reagent database)

f) Provide a hardware control and sensor interface through which feedback and data from sensors in the hardware system are received and signals to operate the hardware emanate

g) Provide an interface to receive signals from sensors set within the electro-mechanical components and the flow channel of the closed loop hardware so that the status and position of the hardware components can be known and flow rates and the position of components in the flow can be sensed. For example, but without restriction the sensors can be mechanical, electrical, photonic, electronic, spectral, wireless, etc.

h) Provide the control signals to coordinate the electro-mechanical actions of the hardware components of the closed loop system either by directly addressing actuators and motors to achieve mechanical movement or by operating through original manufacturers operating systems. For example, but without restriction, LabView (National Instruments) can supply coding and control interfaces

i) Provide a central clock and event scheduler to maintain traceability and provenance of materials in flow within the closed loop system and maintain an internal register to relate assay data to data associated with specific chemical components. This clock also provides synchrony with events arising in the Reaction Design Module (7)

j) Provide a pre-conceived repertoire of control sequences to achieve certain hardware events and routines. Examples include, but are not limited to, retrieving reagents from a known location, introducing reagents into the flow stream at a fixed time and rate, adjusting flow rate to achieve certain objectives within a flow distance, producing flow gradients by variation of pump rates, system maintenance cycles to flush and clean surfaces, diluting analytes by adding buffer in a predetermined ratio prior to assay

k) Provide a translation of the requests from the knowledge-based rationalization and prediction software to the master hardware controller. These requests include the selection of reagents that can be used in the synthesis of a structure predicted to have biological activity by virtue of its ability to mimic the fields described by the knowledge-based kernel as being associated with activity

l) Provide a set of control instructions designed to optimize the performance of the closed loop system based on the data from sensors and any component within the loop (e.g. adjusting flow rates and temperatures and other conditions of assay within the flow assay device, to bring the saturation curve of the assay into the instruments reading range

m) Translate the data obtained from the hardware into a format which can be used by the Reaction Design System (7). In this respect the control computer may draw on other data arising from on-line measurements taken at any location or time point in the closed loop.

As an example, but without restriction, supplementary information may be useful to provide a quantitative or semi-quantitative assessment of the concentration of solutions used in the biological assay to provide values that can be satisfactorily compared to achieve a ranking of the various chemical materials prepared during a multi-cycle experiment on the basis of biological activity. In such a case, at step (3) of the closed loop system described above, the concentration of selected components of the resolved mixture would be quantitatively assessed. As examples, but without restriction, quantification of small samples removed from the closed loop could be assessed through techniques such as, but not exclusively, nuclear magnetic resonance, electrical impedance, ultra violet spectroscopy or chemiluminescent nitrogen detection (CLND), and the like.

Those of skill in the art will appreciate that not all functions need be present in the Realization System for the system to accomplish many of the aspects of the present invention. In general, then, the present invention includes methods of de novo iterative synthesis, systems capable of carrying out the methods, and the synthesized compounds produced by operation of the methods and systems.

### EXAMPLES

### Example 1

### The Realization System

There are many ways of constructing a Realization System. The invention, the “Reaction Design System”, as contemplated herein, is essentially device independent and there is no reason why it could not instruct a human workforce. However, experience has shown that in human hands and at human manual scale this type of strategy is too slow and expensive to provide an economic or timely solution for the evaluation of screening hits from many chemical families to identify the most promising lead series although its principles are used in late stage optimization of leads where only a relatively small number (e.g. 2 or 3) lead series are under consideration.

Thus the notion of exploring in a divergent manner all of the chemotypes presented by the hits identified in a high throughput screening run as starting points for biological data driven chemical programs to identify novel chemotypes not present in the screening collection is beyond current manual and automated methodology. However, a micro-scaled system can achieve this goal because of its speed, frugality with respect to consumption of resources and minimal transfer losses relative to macro methods. Because of the greater choice of more thoroughly tested alternatives the presented execution of the invention provides significantly improved drug leads within a shorter time and with less risk of attrition in development.

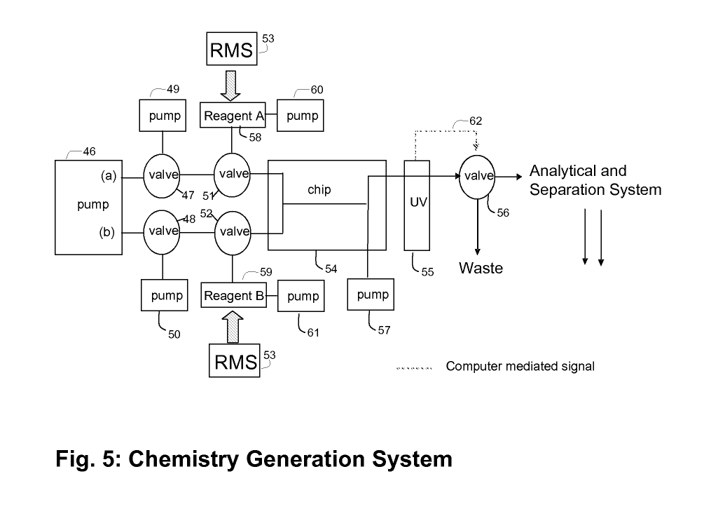

A schematic of the working hardware of a Chemistry Generation System that can be assembled from commercially available components is shown in FIG. 5. This system follows the principles of the generalized Realization System shown in FIG. 4 but varies in detail.

A pump (46) delivers a constant flow of a working solvent to 2 channels (a) and (b) passing through valves (47) and (48), which are turned to shut off flow from pumps (49) and (50), on through valves (51) and (52), which are closed to flow from the Reagent Management Systems (RMS) (53), then on through the chip (54), the UW detector (55) to valve (56) which is set to direct the flow to waste. Throughout this process pump (57) is set to near zero flow. A Reagent Management System (RMS) (53) provides access to a large palette of reagents, two of which are shown here (reagent A and Reagent B) which are delivered by the RMS in solution to receptacles (58) and (59) respectively. Valves (51) and (52) permit loop introduction of a set volume of reagent into the flow. This action is effected by interrupting the flow for a short interval (<1 sec) from pump (46) whilst turning on pumps (60) and (61) for the same interval, thus maintaining an essentially constant flow through the chip (54). Here the ability to simultaneously introduce two reagents (A and B) is shown, but clearly the number of reagent introduction systems can be altered.

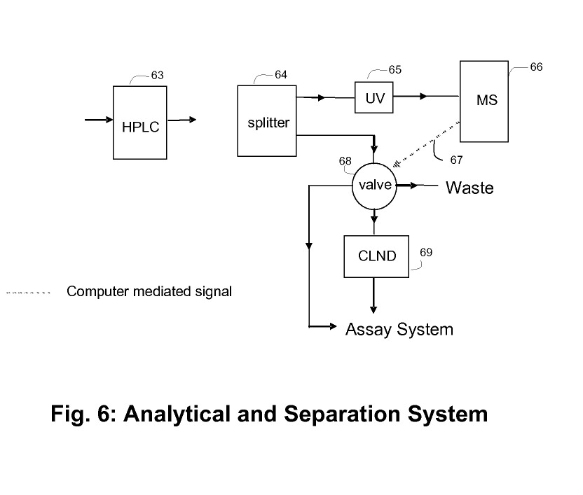

Under control of the computer, solutions of reagents A and B are introduced into the flow via valves (51, 52) to form “slugs” or “plugs” in the flow. These meet at the T-junction on the chip (54) to give a single plug of reaction mixture. During flow along the main channel of the chip the reaction reaches completion and the solution containing the plug flows through a detector (shown as a UV detector (55)) to valve (56). The UV detector allows the detection of the reaction mixture plug in the stream and provides a trigger signal (62) through the computer to operate valve (56) to slice the reaction mixture slug from the flow to be passed to the Analytical and Separation System shown in FIG. 6. Based on information from sensors placed in the Analytical and Separation System, Pump (57) may provide a dilution stream using a solvent compatible with HULK technology. If necessary pumps (49) and (50) perform a bidirectional system flush between reaction runs.

An Analytical and Separation System that can be fed directly by micro-bore silica tubing from the Chemical Generation System described in FIG. 5 is shown schematically in FIG. 6.

The flow from Valve (56) (FIG. 5) which serves as a loop injector, enters the HULK system (63). This HULK system serves to separate the components on the basis of their different retention times in a micro-column filled with a suitable chromatographic medium within the instrument. The different components of the reaction mixture are time-resolved within the HULK system and the different components emerge serially from (63) over a short period of time (0-5 min) to enter the splitter (64) which creates 2 flow streams. The ratio of the flow streams from the flow splitter (64) is set to provide a minimal flow to the UV detector system (65) only sufficient to provide reliable detection by the UV system (65) and the Mass Spectrometer (MS) (66). The UV detector (65) is set to provide warning of the arrival of a plug of a separated UW detectable material so that a sample can be injected into the mass spectrometer (66) to provide a molecular weight (or molecular weight indicator) as a means to further identify that compound in addition to its retention time in the column of the HULK (63). The Mass Spectral data are monitored by the computer system in order to inform the decision of how valve (68) should be operated. The options are:

(i) to waste because the reaction component is confirmed to be of no interest (i.e. unchanged reactant; unwanted side product; etc)

(ii) to a Chemiluminescent Nitrogen Detector (69) (or alternatively an Evaporative Light Scattering Detector, or other quantification device) to provide the quantification data (i.e. sample concentration) required for assay.

(iii) directly to the Assay System, because data from the UW system has given adequate quantification. This provides for a shorter cycle time as when in use, CLND would represent the rate limiting technology.

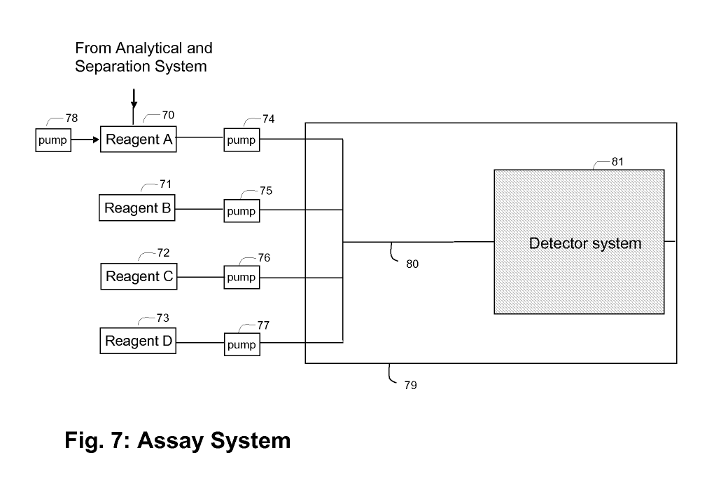

An Assay System that can be fed directly by micro-bore silica tubing from the Analytical and Separation System described in FIG. 6 is shown schematically in FIG. 7. Again, this system follows the principles of the general Realization System shown in FIG. 4 but varies in detail.

The flow from Valve (68) of the Analytical and Separation System is directed into a reservoir (70) marked “Reagent A” which is the test substance at known concentration. An indefinite number of other reservoirs (Reagents B,C,D . . . ) (71, 72, 73) contain the other materials required to conduct an assay. A set of pumps (74, 75, 76, 77) are controlled by a computer to deliver these reagents in the required amounts to create a concentration gradient of the test substance across the range of pharmaceutical interest (from picomolar to over micromolar) with corresponding changes in the quantitative delivery of other assay components so that viable and comparable assay conditions are maintained throughout. A constant flow rate is also maintained. Such a gradient is beyond the dynamic range of current pump technology, therefore the gradient is constructed from several gradients each using a different maximum concentration of the test substance. Concentrations are adjusted by means of a dilution pump (78) pumping buffer.

The flows meet at “T” mixers on the chip (79) as required by the assay and the final assay mixture flows through the main channel (80). A detection system (e.g. a Microscope or an automated equivalent) (81) detects an appropriate confocal volume within the channel where a stable and meaningful reading can be taken.

A commercial instrument made by Genapta Ltd, (William James House, Cowley Road Cambridge, CB4 OWX, United Kingdom) may perform many of the functions as required by the invention. In particular, it provides fully automated detection and thus obviates the need for a microscope and human observer. The Genapta flow assay system enables ligand binding and functional activity measurements in glass channels with dimensions approximately 20×15 microns. It has a temperature controlled stage to hold the chip and 4 channel pump and valve system to manage fluid flows and generate the concentration gradient required to determine the concentration required for a half-maximal effect (or some other standard measuring point). The chip is mounted on a closed loop motion control system which can move the optical spot relative to the channel over a 20 mm span down to an accuracy of less than 2 microns, thus enabling accurate mapping of the chemical and biological changes as the fluids interact. The system uses Genapta's focusing system (see, e.g., WO03/048744) to locate the channels and the focal depth which obviates the need for an optical microscope or manual intervention. The Genapta optical head simultaneously interrogates a 30 fl sample with two colors of laser light and fluorescence intensity, fluorescence polarization or Fluorescent Resonant Energy Transfer (FRET) is used to determine ligand binding. Sensitivity ranges down to as few as 400 molecules in the sample volume at any one time.

This level of detection within assays shows that the target quantity of 1 microgram of product from the micro chemistry generator is well in excess of requirements and even with several assays in place there is a redundancy that allows for reactions with very low yields to furnish sufficient material. Because of miniaturization the time required per assay for in vitro assays is in the order of a few minutes and along with the HULK set the lower limit of cycle time to a similar value.

### Example 2

### The Reaction Design System

In assessing the performance of the System no example can be definitive because there is no end to the optimization with an almost infinite universe of chemical opportunity. If the “lead” is the best molecule available at any moment (by whatever parameters we are judging) then identifying a better compound displaces it. The new compound then becomes the “lead”. To provide an example, we impose a limit such as time, number of compounds, number of iterations, etc., knowing that due to the iterative nature of the process, whatever is best at a moment may not be the best ultimately if further iteration brings that lead series to a blank wall. We may need to return to choose an alternative to an earlier choice from other molecules showing potential.

In fact, it is the nature of the algorithm that we have a cohort of molecules proceeding forward which we select from according to some rules of priority. The best indication that the invention does what is expected:

(a) demonstrate that there is a cohort of diverse molecules—this establishes that the method will have value to the user by identifying a wider range of patentable structures than present methods and thus providing more security.

(b) demonstrate that the cohort pool can be enriched by iteration (i.e., no plateau was reached) and therefore because the pharmacophore continues to improve, the type of structure that can be proposed to fit the pharmacophore will change. (i.e. that we are not in a stasis, but can go forward to improved compounds.)

(c) Demonstrate that in respect of any assay, that it does not provide an elite solution for that assay but that several chemotypes can be identified so that if the desired activity profile is determined by the results of several assays, there are multiple chemotypes to present distinctly different opportunities to achieve an optimal combination of properties. (Note that no activity would have to be maximal or minimal in a chemotype—it just needs to have sufficient presence or absence of the desired characteristics and adequate therapeutic activity)

(d) demonstrate that the improvement relative to effort can be substantial and superior to current methods.

Thus, the speed of delivery of compounds and route exploration will be many times faster than macro iterative endeavors. Additionally, synthesizing a microgram will embarrass supply lines to a lesser degree than synthesizing a gram, thus the present invention will consume less biological reagents than array methods.

### Example 3

### The Reaction Design System

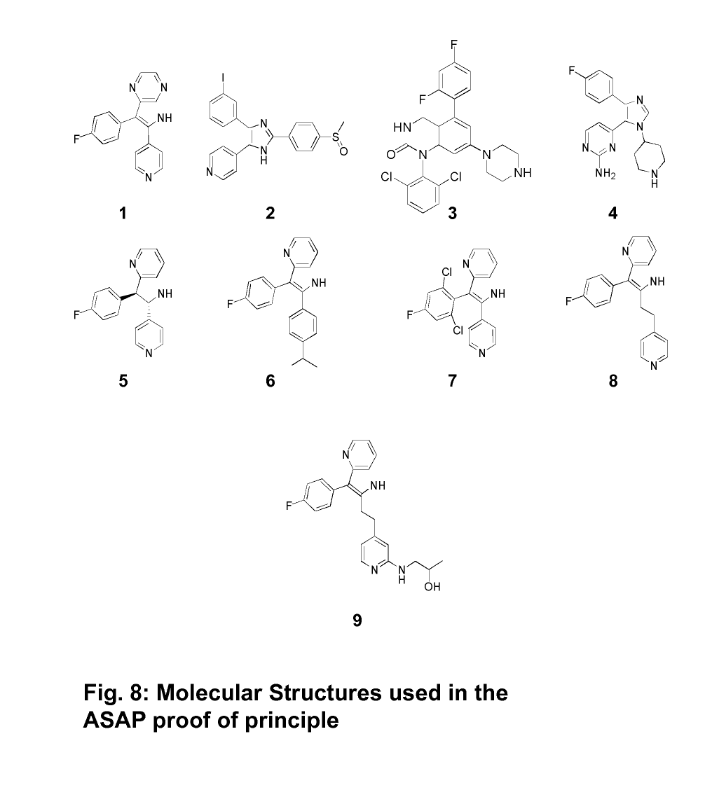

a) An ASAP system process (FIG. 8)

A field scoring-based pharmacophore of the inhibitor (molecule 1), with low activity against p38 MAP kinase, was used as a starting seed onto which three other field pharmacophores of known structurally diverse inhibitors (molecules 2-4) were aligned. The ASAP module of the Reaction Design System then combined the information from all four ligands to produce a refined starting field pharmacophore from which it indicated a list of possible molecules to be made and tested. Molecules 5-8 were at hand from the literature and structures and biological results of these four compounds could be used as if they had exited from the Realization System. The compounds were fed back into the Reaction Design System to further modify and refine the model. More new compounds were derived from this second generation model by the ASAP and passed to the Realization System. One of these (molecule 9) was found to have been reported in the literature and showed a 60-fold increase in activity over the starting compound (molecule 1) on exit from the Realization System.

b) The ASAP module

To further demonstrate the power of the method a test collection of 2500 potentially active diverse chemotypes was designed. 75% proved to be synthesizable in a single attempt. These realized compounds were assayed. A seed molecule NOT present in the test collection was used to generate a pharmacophore using field scoring which was then used to identify potential bioisosteres from within the collection. These were prioritized in respect of the closeness of their pharmacophores to the lead pharmacophore. The closest fitting 5% were then selected for synthesis as a first iteration. Of these about the same proportion (75%) were synthesizable and these realized compounds were assayed. The data were returned to the program to improve the selection pharmacophore. This was used to again select the 5% closest fitting pharmacophores of the test collection. The new compounds entering this group were synthesized as the second iteration. The ratio of first-time un-synthesizable compounds was unaffected by these additions and was again about 25%.

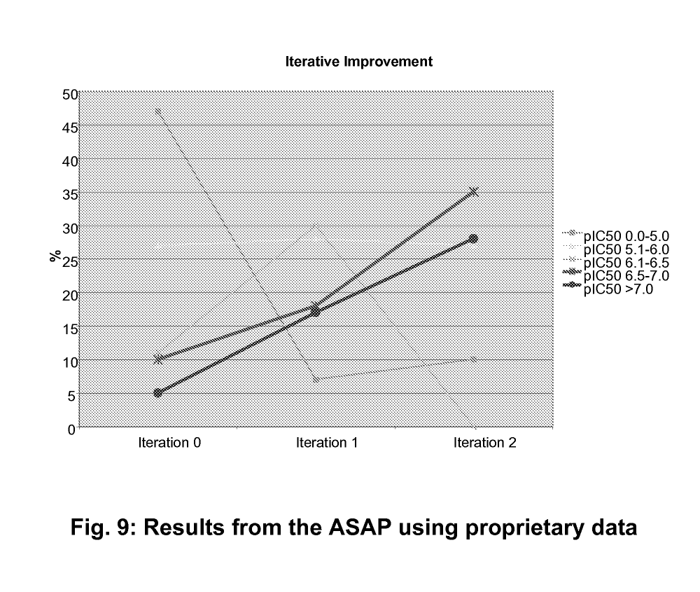

The cumulative results are shown in FIG. 9. The proportions of each of the more potent molecules in the selection (i.e. pIC50>7.0 and pIC50 6.5-7.0-shown bold) was substantially increased by each iteration whereas the proportion of the group with lowest activity (pIC50 0-5.00) was substantially reduced.

It will be apparent to persons skilled in the art that numerous enhancements and modifications can be made to the above described apparatus without departing from the basic inventive concepts. All such modifications and enhancements are considered to be within the scope of the present invention, the nature of which is to be determined from the foregoing description and the appended claims. Furthermore, the preceding Examples are provided for illustrative purposes only, and are not intended to limit the scope of the invention. All references cited herein are expressly incorporated by reference herein.

1. A method of de novo iterative synthesis, comprising the steps of:
a) selecting a candidate compound having desired pharmacophoric fit with a seed structure;
b) synthesizing the candidate compound;
c) assaying the synthesized compound, and comparing the synthesized compound to the seed structure to determine whether the synthesized compound has synthetically desirable properties, wherein if the synthesized compound does not have synthetically desirable properties, step a) is repeated for a new candidate compound, and if the synthesized compound does have synthetically desirable properties, then step d) is performed; and
d) iterating steps a) through c) wherein the synthesized compound is used as the seed structure of step a), until exhaustion.

2. The method of claim 1, wherein the candidate compound is selected based on additional criteria selected from the group consisting of availability of reagents and viability of synthetic routes.

3. The method of claim 1, wherein the pharmacophoric fit is assessed using field scoring.

4. The method of claim 1, wherein synthetically desirable properties comprise data selected from the group consisting of demonstrating greater bioactivity, and providing information regarding allowed, disallowed, or novel regions of space.

5. The method of claim 1, wherein exhaustion occurs when subsequent synthesized compounds are not superior to previous synthesized compounds for at least 3 iterations

6. The method of claim 1, wherein step c) is performed using ASAP.

7-10. (canceled)

11. A system for de novo iterative synthesis, comprising
a) a reaction design system; and
b) a realization system;
wherein the reaction design system and realization system are capable of passing information bilaterally via computer interaction to achieve de novo iterative synthesis.

12. The system of claim 11, wherein the reaction design system comprises:
a) a database of field mapped compounds;
b) a structure fitting pharmacophore generator;
c) a computer and software; and
d) optionally, a fragment database.

13. The system of claim 12, wherein the reaction design system generates a list of candidate compounds selected from the database of field mapped compounds, and outputs instructions to the realization system to synthesize a particular candidate compound.

14. The system of claim 11, wherein the realization system comprises
a) a microfluidic apparatus comprising a pump, reagents, reagent ports, a reaction zone, a plurality of sensors, a flow conditioner, and a flow assay system;
b) a computer and software;
wherein the realization system receives input from the reaction design system instructing the synthesis of a candidate compound, and wherein the realization system is capable of synthesizing and assaying the candidate compound.

15. A molecule synthesized by the method of claim 1.

16. A molecule synthesized by the method of claim 4.

17. A molecule synthesized by the method of claim 6.

18. A molecule synthesized by the system of claim 11.

19. A molecule synthesized by the system of claim 12.

20. A molecule synthesized by the system of claim 14.

21. (canceled)
